(19) United States
(12) Patent Application Publication
(10) Pub. No.: US 2013/0144278 A1
(43) Pub. Date: Jun. 6, 2013

(54) Devices and Methods for Multispot Scanning

(71) Applicants: Michael Papac, North Tustin, CA (US); Michael J. Yadlowsky, Sunnyvale, CA (US); John Christopher Huculak, Mission Viejo, CA (US)

(72) Inventors: Michael Papac, North Tustin, CA (US); Michael J. Yadlowsky, Sunnyvale, CA (US); John Christopher Huculak, Mission Viejo, CA (US)

(21) Appl. No.: 13/692,260

(22) Filed: Dec. 3, 2012

Related U.S. Application Data
(60) Provisional application No. 61/567,439, filed on Dec. 6, 2011.

(51) Int. Cl. A61F 9/008; A61B 3/10

(52) U.S. Cl. CPC A61F 9/00821; A61B 3/10
USPC 606/4; 351/205

(57) ABSTRACT

An ophthalmic endoprobe system comprises an optical fiber configured to transmit light energy along an optical axis. The system further comprises a first scanning element rotatable relative to the optical fiber and arranged to receive at least a portion of the transmitted light energy. The first scanning element includes a diffractive optical element. The system also comprises a second scanning element rotatable relative to the first scanning element and arranged to receive at least a portion of the transmitted light energy from the first scanning element.

### PRIORITY CLAIM

This application claims the benefit of priority of U.S. Provisional Patent Application Ser. No. 61/567,439 titled “Devices and Methods for Multispot Scanning”, filed on Dec. 6, 2011, whose inventors are Michael Papac, Michael J. Yadlowsky, and John C. Huculak, which is hereby incorporated by reference in its entirety as though fully and completely set forth herein.

### FIELD

The present application relates to a probe for use in ophthalmic procedures and more particularly to a multispot laser probe for use in photocoagulation.

### BACKGROUND

Anatomically, the eye is divided into two distinct parts—the anterior segment and the posterior segment. The anterior segment includes the lens and extends from the outermost layer of the cornea (the corneal endothelium) to the posterior of the lens capsule. The aqueous humour fills the space between the lens and the cornea and helps maintain intraocular pressure. The posterior segment includes the portion of the eye behind the lens capsule. The posterior segment extends from the anterior hyaloid face to the retina. The retina is a light-sensitive tissue that lines the inner surface of the eye. Blood vessels that supply the retina form two circulations, the uveal and the retinal circulations. Both circulations are supplied by the ophthalmic artery. Diseases affecting the retina include diabetic retinopathy and macular degeneration. Diabetic retinopathy is a condition that occurs when high levels of blood glucose damage the blood vessels of the retina, causing blood leakage. Macular degeneration is a condition that occurs when abnormal blood vessels grow under the retina. These vessels may leak and lead to blurred vision or blindness. These and other types of retinal diseases may be treated with photocoagulation therapies. Photocoagulation involves the precise and concentrated application of laser energy to cauterize or “burn” leaking, damaged, weakened, or otherwise abnormal blood vessels. Panretinal photocoagulation is a type of photocoagulation procedure that involves the application of multiple burns to a region of the retina. Existing endoprobes, used in photocoagulation procedures, generally provide a fixed beam emission which requires an ophthalmic surgeon to turn a laser beam on and off, in rapid fire succession with a foot pedal, as the beam is manually scanned across the retinal surface to create a one or two dimensional array of photocoagulated laser burn spots on the retina. Systems and methods are needed to shorten the duration and improve the accuracy of retinal photocoagulation procedures.

### SUMMARY

Further aspects, forms, embodiments, objects, features, benefits, and advantages of the present invention shall become apparent from the detailed drawings and descriptions provided herein.

In one embodiment, an ophthalmic endoprobe system comprises an optical fiber configured to transmit light energy along an optical axis. The system further comprises a first scanning element rotatable relative to the optical fiber and arranged to receive at least a portion of the transmitted light energy. The first scanning element includes a diffractive optical element. The diffractive optical element may be, for example, a holographic optical element. The system also comprises a second scanning element rotatable relative to the first scanning element and arranged to receive at least a portion of the transmitted light energy from the first scanning element.

In another embodiment, a method of laser photocoagulation comprises transmitting light energy along an optical axis of an optical fiber. The method also includes rotating a first scanning element relative to the optical fiber. The first scanning element includes a diffractive optical element. The method also includes rotating a second scanning element relative to the first scanning element and transmitting at least a portion of the light energy through the first and second scanning elements to produce a scan pattern on a target tissue.

In still another embodiment, an ophthalmic laser endoprobe system comprises a laser configured to generate light energy and an optical fiber configured to transmit the light energy along an optical axis. The system further comprises a collimating optical component arranged to receive and collimate the transmitted light energy. The system further includes first diffractive scanning element configured as a cylindrical plate and rotatable about the optical axis relative to the optical fiber. The first diffractive scanning element is arranged to receive at least a portion of the collimated light energy. The system also includes a second scanning element rotatable about the optical axis relative to the first scanning element. The second scanning element is arranged to receive at least a portion of the collimated light energy from the first diffractive scanning element.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIGS. 1a, 1b, and 2 are diagrams of an endoprobe system according to one embodiment of the present disclosure.

FIG. 3 is a diagram of a distal portion of an endoprobe system according to another embodiment of the present disclosure.

FIG. 4 is a diagram of an endoprobe system according to another embodiment of the present disclosure.

FIG. 5 is a diagram of an endoprobe system according to another embodiment of the present disclosure.

FIG. 6 is a diagram of an endoprobe system according to another embodiment of the present disclosure.

FIG. 7 is a diagram of an endoprobe system according to another embodiment of the present disclosure.

FIG. 8 is a diagram of an endoprobe system according to another embodiment of the present disclosure.

FIG. 9 is flowchart describing a method of operating an endoprobe system according to another embodiment of the present disclosure.

### DETAILED DESCRIPTION OF PREFERRED EMBODIMENTS

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

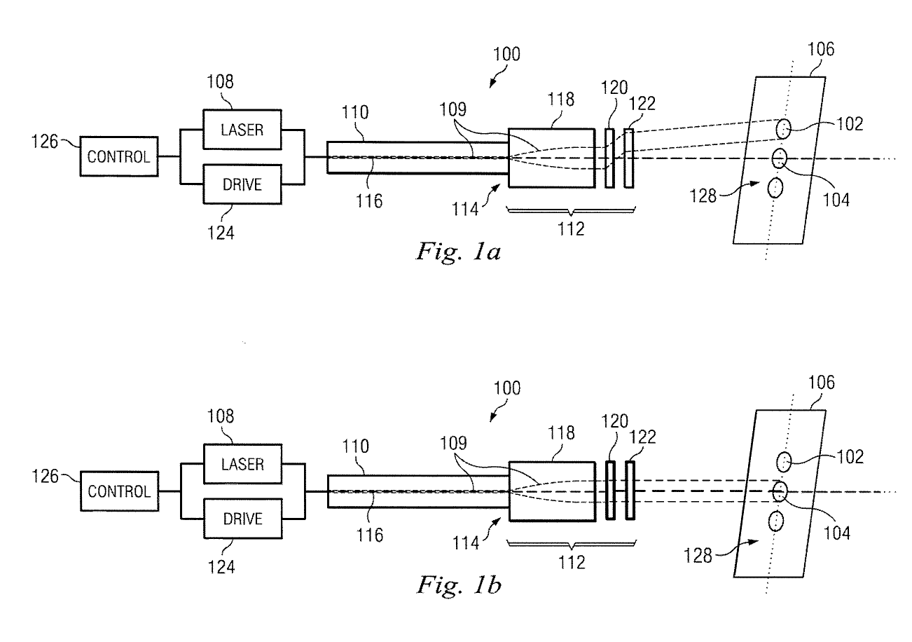

FIG. 1a is a diagram of an endoprobe system 100 used in therapeutic applications to deliver light energy to multiple spots or locations 102, 104 at a tissue site 106. The endoprobe system 100 includes a laser 108 operable to generate light energy 109 in the form of a light beam. The laser may be a diode laser, a gas laser, a solid state laser, or any other laser device for the stimulated emission of light energy. The generated light energy 109 is transmitted via a transmission device 110 to a scanning apparatus 112 located at or near a distal end 114 of the transmission device. The transmission device 110 may be, for example, an optical fiber or other type of optical waveguide. The optical fiber 110 may transmit the generated light energy 109 along an optical axis 116. The transmission device and the scanning apparatus may be housed in a handheld endoprobe instrument body. In one embodiment, the instrument body is approximately 75 millimeters (mm) in length and approximately 12 mm in diameter. In other embodiments, instrument bodies suitable for handheld use may have larger or smaller dimensions.

The scanning apparatus 112 includes an optical component 118 that collimates light energy 109. The collimating optical component 118 may also expand or converge the light energy prior to collimation. Suitable collimating optical components may include cylindrical gradient index (GRIN) lenses, ball lenses, or aspherical lenses. The collimating optical component 118 may be attached directly to or spaced apart from the optical fiber 110. In this embodiment, the collimating optical component 118 is aligned about the optical axis 116.

The scanning apparatus 112 may also include scanning optical elements 120, 122. The scanning optical elements 120, 122 in this embodiment are diffractive optical elements. Suitable diffractive optical elements may include for example, ruled diffraction gratings, holographic diffraction gratings, volume phase holographic diffraction gratings, and/or digital planar holographic gratings. The first scanning element 120 deflects the beam of light energy 109 off axis 116 so that the exiting beam of light energy is translated and angled relative to the entering beam of light energy. The second optical element 122 may provide further deflection, compensatory deflection, and/or deflection in a direction orthogonal to that of the optical element 120. Each of the optical elements 120, 122 may change the direction of the received light energy 109 by a predetermined amount. Used in combination, the optical elements 120, 122 may deflect the light energy 109, for example, between approximately +/−20 degrees from the optical axis 116. The diffractive optical elements 120, 122 are independently rotatable relative to each other and about the optical axis 116. Controlling the rotation of the optical elements 120, 122 relative to each other allows a user to control the direction of the transmitted light energy 109 to form a scan pattern on the tissue site 106.

In this embodiment, the diffractive optical elements are aligned about the optical axis 116. In this embodiment, the optical elements 120, 122 are cylindrically shaped with the cylindrical faces of optical element 120 positioned approximately parallel to the cylindrical faces of the optical element 122. Also in this embodiment, the optical elements 120, 122 have approximately the same diffractive properties. In alternative embodiments, the optical elements may be angled relative to each other or may have different diffractive properties. As compared to refractive prisms or lenses, the cylindrical diffractive optical elements may be more compact, easier to align, and less expensive to produce. In still other alternative embodiments, a single optical component may combine the functions of the collimating optical component and one or more of the scanning optical components.

The endoprobe system 100 also includes a drive system 124 which actuates the optical elements 120, 122. The drive system may include motors, gears, and other mechanical, electrical, and/or electromechanical components for driving the rotation of the optical elements 120, 122. The endoprobe system 100 also includes a control system 126 that receives instructions from a user or from a computer to control and synchronize the laser 108 and the drive system 124. The control system 126 may receive user input from, for example, a button or footswitch. Although not shown, in some embodiments, synchronized mechanical beam choppers or optical attenuators, located within the console of the laser or within the endoprobe handpiece, may also or alternatively be used to synchronize the exposure of the laser with the configuration of the scanning optical elements.

In FIG. 1 a, the optical elements 120, 122 are arranged in a first configuration relative to each other such that the light energy 109 transmitted through the optical elements is delivered to location 102 on the tissue site 106. The light energy 109 may cause photocoagulation of the tissue at the location 102. In Fig. lb, the optical elements 120, 122 have been counter-rotated, that is rotated the same angular distance in opposite directions, to a second configuration. In this second configuration, light energy 109 transmitted through the optical elements 120, 122 is delivered to the location 104 on the tissue site 106. Additional counter rotation may produce a configuration of the optical elements 120, 122 to deliver light energy 109 to a location on the opposite side of the linear scan pattern as location 102 (e.g., after 180 degree rotation from the original optical element positions). Further counter rotation of the elements may move the light in the opposite direction back to the original location 102 (e.g., after a complete rotation of each element), and the cycle may be repeated. When the optical elements 120, 122 are counter rotated, the light energy 109 may be delivered to locations that form a linear scan pattern 128 as shown in FIGS. 1a and 1b. In one embodiment, the laser 108 may be pulsed as the optical elements 120, 122 are held stationary at different counter rotated configurations. In alternative embodiments, the optical elements 120, 122 may be continuously counter rotating while the laser 108 is pulsed. In still another alternative embodiment, the laser 108 may be continuously operated as the optical elements 120, 122 are continuously counter rotated or counter rotated and stopped in specific configurations. Shorter pulses and stationary optical elements may result in more uniform and intensive delivery of light energy to discrete locations 102, 104. Longer pulses or continuous delivery of light energy together with continuously rotating optical elements 120, 122 may result in the distributed delivery of light energy between locations 102, 104. The control system 126 may control and coordinate the laser 108 and the drive system 124. For example, the control system 126 may control the timing and length of the laser pulses and may control the direction and speed of the rotation of the optical elements 120, 122 via the drive system 124.

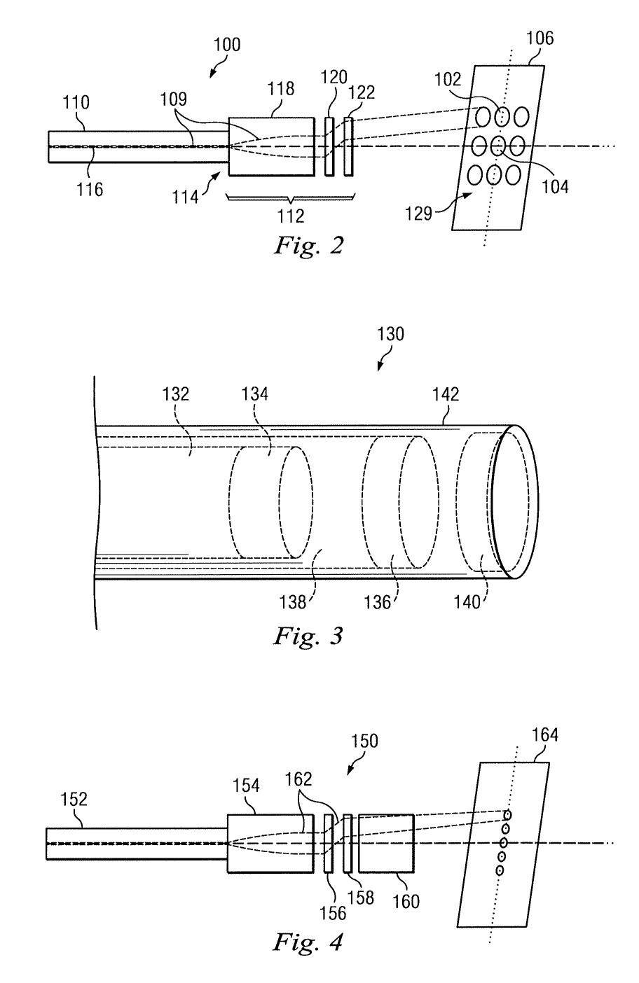

As shown in FIG. 2, the endoprobe system 100 may also be used to generate a two dimensional scan pattern 129 at the tissue site 106. Two dimensional scan patterns may be generated by rotating one or both of the optical elements 120, 122 through a series of predetermined configurations with respect to each other. As described for FIGS. 1a and 1b, precise counter rotation of the optical elements 120, 122 may generate the linear scan pattern 128. As shown in FIG. 2, the two dimensional scan pattern 129 can be generated by altering the rotation of the optical elements with respect to each other to change the direction of the light energy 109 through a two dimensional pattern. In various embodiments, the optical elements may be rotated in the same direction or in opposite directions. In various embodiments, the optical elements may be rotated at the same or at differing speeds. In various embodiments, the optical elements may be rotated continuously or may be rotated and stopped at predetermined locations. The generated two dimensional scan patterns may be generally rectilinear patterns or may be more arbitrary or complex patterns implemented by the use of software with the controller 126.

FIG. 3 is a diagram of a distal portion of an endoprobe system 130 according to one embodiment of the present disclosure. The endoprobe system 130 may include the same types of components described above for endoprobe system 100. In this embodiment, an optical fiber 132 delivers light energy to a collimating optical component 134. A diffractive optical element 136 is coupled to a tube 138, and a diffractive optical element 140 is coupled to a tube 142. Each of the tubes 138, 142 are concentric with the optical fiber 132. The tubes 138, 142 are part of the drive system associated with the endoprobe system 130 and may be actuated to rotate independently of each other or in a coupled manner.

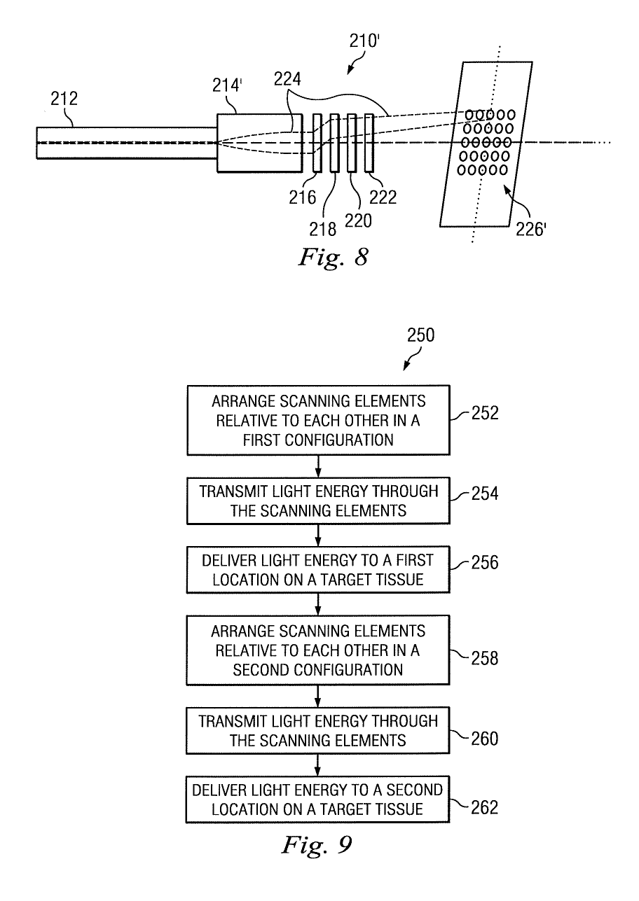

FIG. 4 is a diagram of an endoprobe system 150 according to another embodiment of the present disclosure. The endoprobe system 150 includes an optical fiber 152, a collimating optical component 154, and scanning optical elements 156, 158. These components of the endoprobe system may be the same as or substantially similar to the corresponding components of the endoprobe system 100. The endoprobe system 150 further includes a condensing lens 160 located distally of the optical elements 156, 158. In this embodiment, light energy 162 is transmitted through the optical fiber 152 and collimated by the optical component 154. After the collimated light energy 162 is transmitted through and deflected by the rotatable optical elements 156, 158, the condensing lens 160 focuses the light energy. As compared to the more collimated light delivered in the embodiment of FIG. 1a, the focused light energy 162 may deliver a more intense light energy to more compact locations at the tissue site 164. Suitable condensing lenses may include biconvex and plano-convex lenses. Alternatively, a focused beam of light energy may be generated without the use of a condensing lens. For example, the collimating optical component may be replaced with a primary lens designed with more optical power than is required for collimation. In this alternative, the primary lens may focus the light energy distally of the optical elements 156, 158. (e.g., see FIG. 8).

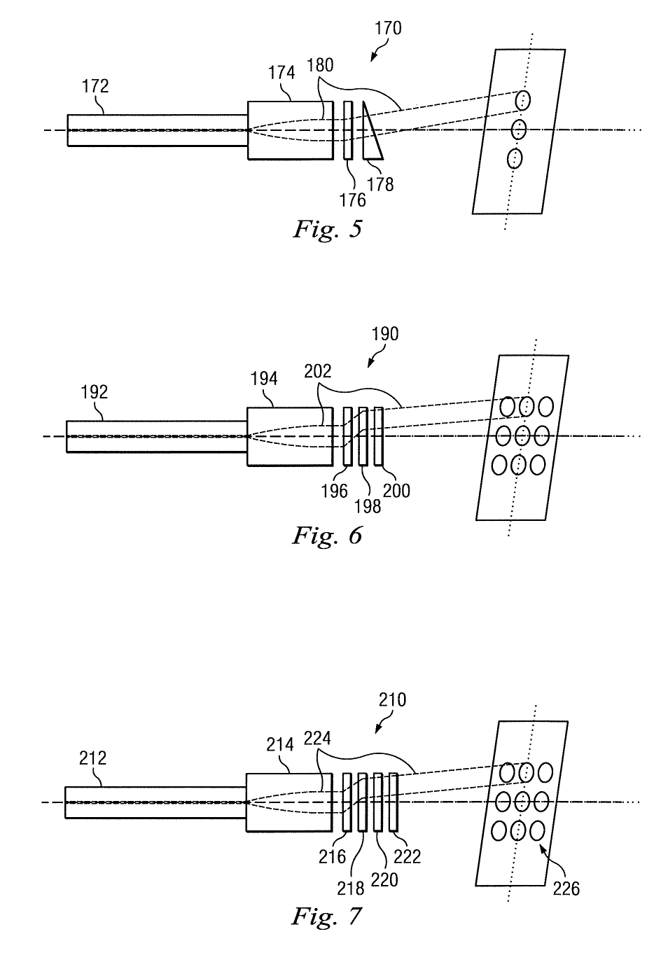

FIG. 5 is a diagram of an endoprobe system 170. The endoprobe system 170 includes an optical fiber 172, a collimating optical component 174, and scanning optical elements 176, 178. Scanning optical element 176 may be a diffractive optical element as described above. In this embodiment, the scanning optical element 178 is a refractive optical element positioned distally of the diffracting optical element 176. The refractive optical element 178 receives the diverted light energy 180 from the optical element 176 and further changes the direction of the light energy depending upon the configuration of the optical element 178 relative to the optical element 186. In use, the refractive optical element 178 and the diffracting optical element 176 may be counter rotated or rotated by differing amounts so that light energy 180 passing through the optical elements may be steered through a linear or two dimensional scan pattern. The other components of the endoprobe system may be the same as or substantially similar to the corresponding components of the endoprobe system 100.

FIG. 6 is a diagram of an endoprobe system 190. The endoprobe system 190 includes an optical fiber 192, a collimating optical component 194, and three scanning optical elements 196, 198, 200. Scanning optical element 196, 198, 200 may be diffractive optical elements as described above. The use of three scanning elements may provide greater variety and flexibility in the generated scan patterns. Further, three scanning elements may simplify or provide more versatility in the scanning element drive requirements and the laser pulse synchronization. In this embodiment, any two of the scanning elements 196, 198, 200 may be continuously rotated at constant velocities while the rotation of the third scanning element is controlled to position the third scanning element at specific rotational positions relative to the optical fiber 192. As the third scanning element is cycled to each of the specific positions, the laser pulse delivers light energy 202 to generate a linear or two dimensional scan pattern. In alternative embodiments, the rotations of each of the scanning optical elements 196, 198, 200 may be synchronized and controlled.

FIG. 7 is a diagram of an endoprobe system 210. The endoprobe system 210 includes an optical fiber 212, a collimating optical component 214, and four scanning optical elements 216, 218, 220, 222. Scanning optical elements 216, 218, 220, 222 may be diffractive optical elements as described above. In this embodiment, each of the four scanning optical elements 216, 218, 220, 222 may be rotated at a constant velocity which may be the same or different for each optical element. As the optical elements are rotating, the laser may be synchronized to produce light energy 224 that is transmitted from the distal most optical element 222 as a continuous raster scan. The continuous raster scanning generates a two-dimensional scan pattern 226. Alternatively, instead of operating in continuous rotation at constant speeds, the rotations of one or more of the scanning optical elements 216, 218, 220, 222 may be controlled to stop or slow at predetermined positions synchronized with the laser pulses.

FIG. 8 is a diagram of an endoprobe system 210′ with a configuration similar to that of endoprobe system 210 of FIG. 7. In the embodiment of FIG. 8, a primary lens 214′ is designed with more optical power than is required for collimation. The primary lens 214′ focuses the light energy 224 distally of the distal-most optical element 222. As compared to the more collimated light delivered in the embodiment of FIG. 7, the focused light energy of FIG. 8 may deliver a more intense light energy to more compact locations in a two-dimensional scan pattern 226′ at the tissue site 164.

The systems described in the example embodiments may be used for ophthalmic photocoagulation treatment, however, no limitation of the scope of the disclosure is intended. In other embodiments, the systems described herein may be used for photocoagulation in other internal or external tissue locations in a human or animal body. In still other embodiments, the systems described herein may be used to deliver light energy to multiple locations for the purpose of imaging, illumination, surgical procedures, or other therapeutic and non-therapeutic purposes.

FIG. 9 is a flowchart 250 describing a method of operating an endoprobe system, such as endoprobe system 100, according to an embodiment of the present disclosure. At 252, scanning elements 120, 122 are arranged relative to each other in a first configuration. The first configuration may occur as the scanning elements 120, 122 are in rotational motion or may occur after the scanning elements have been rotated and stopped. At 254, with the scanning elements 120, 122 in the first configuration, the laser 108 is activated and pulsed light energy 109 is transmitted through the scanning elements 120, 122. At 256, the light energy 109 is delivered to the target tissue 106 at location 102. At 258, the scanning elements 120, 122 are arranged relative to each other in a second configuration. The second configuration may occur as the scanning elements 120, 122 are in rotational motion or may occur after the scanning elements have been rotated and stopped. At 260, with the scanning elements 120, 122 in the second configuration, the laser 108 is activated and pulsed light energy 109 is transmitted through the scanning elements 120, 122. At 262, the light energy 109 is delivered to the target tissue 106 at location 104. This process may be repeated with the scanning elements 120, 122 arranged in different configurations to generate a linear or two dimensional scan pattern. When the scanning elements 120, 122 are arranged in generally counter rotated configurations, the generated scan pattern may be generally linear. When the scanning elements 120, 122 are arranged in configurations with varying angular ratios between the scanning elements, the generated scan pattern may be two dimensional, such as a rectilinear scan pattern.

In some embodiments, an ophthalmic laser endoprobe system may include a laser configured to generate light energy, an optical fiber configured to transmit the light energy along an optical axis, a collimating optical component arranged to receive and collimate the transmitted light energy, a first diffractive scanning element configured as a cylindrical plate and rotatable about the optical axis relative to the optical fiber, the first diffractive scanning element arranged to receive at least a portion of the collimated light energy, and a second scanning element rotatable about the optical axis relative to the first scanning element, the second scanning element arranged to receive at least a portion of the collimated light energy from the first diffractive scanning element. In some embodiments, the first diffractive scanning element includes a diffractive grating. In some embodiments, the first diffractive scanning element includes a holographic element.

In some embodiments, the system further includes a focusing optical element arranged to receive at least a portion of the collimated light energy from the second scanning element and produce focused light energy.

As compared to endoprobe systems that produce only a single beam of non-steerable light energy, the embodiments of this disclosure may increase the number of locations at a target tissue site that may be treated while decreasing the time to treat the multiple locations. Further, the intensity of the light energy delivered to each location may be more consistent. Further still, more complicated scan patterns may be generated.

The term “such as,” as used herein, is intended to provide a non-limiting list of exemplary possibilities. The term “approximately” or “about,” as used herein, should generally be understood to refer to both numbers in a range of numerals. Moreover, all numerical ranges herein should be understood to include each whole integer and tenth of an integer within the range.

While various embodiments of the invention have been described above, it should be understood that they have been presented by way of example only, and not limitation.

Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

We claim:

1. An ophthalmic endoprobe system, comprising:
an optical fiber configured to transmit light energy along an optical axis;
a first scanning element rotatable relative to the optical fiber and arranged to receive at least a portion of the transmitted light energy, wherein the first scanning element includes a diffractive optical element; and
a second scanning element rotatable relative to the first scanning element and arranged to receive at least a portion of the transmitted light energy from the first scanning element.

2. The endoprobe system of claim 1, wherein the second scanning element includes a diffractive optical element.

3. The endoprobe system of claim 1, wherein the second scanning element includes a refractive optical element.

4. The endoprobe system of claim 1, wherein the diffractive optical element includes diffractive gratings.

5. The endoprobe system of claim 1, wherein the diffractive optical element includes a holographic optical element.

6. The endoprobe system of claim 1, wherein the diffractive optical element is cylindrical.

7. The endoprobe system of claim 1, further comprising a beam collimating optical component disposed between the optical fiber and the first scanning element.

8. The endoprobe system of claim 7, wherein the beam collimating optical component is a gradient index (GRIN) lens.

9. The endoprobe system of claim 1, further comprising a focusing optical element arranged to receive and focus at least a portion of the transmitted light energy from the second scanning element.

10. The endoprobe system of claim 1, further comprising a third scanning element rotatable relative to the second scanning element and arranged to receive at least a portion of the transmitted light energy from the second scanning element.

11. A method of laser photocoagulation, comprising:
transmitting light energy along an optical axis of an optical fiber;
rotating a first scanning element relative to the optical fiber, wherein the first scanning element includes a diffractive optical element;
rotating a second scanning element relative to the first scanning element; and
transmitting at least a portion of the light energy through the first and second scanning elements to produce a scan pattern on a target tissue.

12. The method of claim 11, further comprising counter rotating the first and second scanning elements at the same angular speed.

13. The method of claim 11, wherein transmitting the light energy includes transmitting a plurality of laser pulses.

14. The method of claim 11, wherein the produced scan pattern includes a linear scan pattern.

15. The method of claim 11, wherein the produced scan pattern includes a two dimensional scan pattern.

16. The method of claim 11, further comprising collimating the light energy before the transmitting at least a portion of the light energy through the first and second scanning elements.

17. The method of claim 11, further comprising focusing the light energy before producing the scan pattern on the target tissue.

18. The method of claim 11, wherein the second scanning element includes a diffractive optical element.

19. The method of claim 11, wherein the second scanning element includes a refractive optical element.

20. The method of claim 11, wherein the diffractive optical element includes diffractive gratings or a holographic optical element.
